(19) United States
(12) Patent Application Publication
(10) Pub. No.: US 2017/0340044 A1
(43) Pub. Date: Nov. 30, 2017

(54) HEADGEAR PADDING WITH MULTIPLE REMOVABLE LINERS

(71) Applicant: Nue Medical Solutions, LLC, Aurora, IL (US)

(72) Inventors: Aris John Balderama Arenas, Aurora, IL (US); Chris Chung-Ming Huang, Hinsdale, IL (US); Paul M. Korzenecki, Naperville, IL (US); Mathew G. Gavronski, Glen Ellyn, IL (US)

(21) Appl. No.: 15/605,894

(22) Filed: May 25, 2017

Related U.S. Application Data
(60) Provisional application No. 62/342,182, filed on May 26, 2016.

(51) Int. Cl. A42B 1/06; A42C 5/02; A42B 1/22; A61B 90/50; A61B 90/53

(52) U.S. Cl. CPC A42B 1/069; A42C 5/02; A42B 1/225; A61B 90/53; A61B 2090/502

(57) ABSTRACT

A padding assembly suitable for the interior of headgear, such as surgical headgear, includes a padding layer with first and second sides. An attachment layer is provided on the first side of the padding layer for attaching the padding assembly to the headgear. A plurality of comfort layers are stacked one upon the other on the second side of the padding layer. Each comfort layer includes an outer contact layer formed of a comfort material, and an adhesive layer opposite the contact layer for securing each comfort layer to an underlying comfort layer. Each comfort layer may be removed upon becoming soiled to expose the underlying clean comfort layer for a subsequent use.

### CROSS-REFERENCE TO RELATED APPLICATION

This application claims the benefit of and priority to U.S. Provisional Patent Application No. 62/342,182, filed May 26, 2016, the entire contents of which are hereby incorporated by reference herein.

### BACKGROUND

The present disclosure relates to padding, and more specifically, to a padding assembly with multiple removable liners that may be removed one-by-one to expose a clean user-engaging surface with each use. The padding assembly is particularly well suited for use in a surgical environment in combination with various types of surgical headgear.

Padding assemblies are known for use in headgear to improve the level of fit and comfort of a wearer. Many padding assemblies include a conforming layer formed of a relatively compliant material that conforms to the unique contours of an individual wearer's head. Padding assemblies may also include a comfort layer overlying the conforming layer and positioned for direct contact with the wearer's head. The comfort layer may be selected of a material that relatively soft to the touch and/or moisture absorbing to improve comfort to the wearer and to absorb sweat that may accumulate under the padding assembly during use. Such known padding assemblies may be permanently attached to a piece of headgear, or in some cases padding may be removable for washing or for replacement with a new padding assembly when the padding assembly becomes soiled to a degree where further use is undesirable.

### SUMMARY

In some aspects, a padding assembly releasably attachable to surgical headgear is provided and includes a padding layer formed of a compliant solid state gel material, an attachment layer coupled to a first side of the padding layer and configured for attachment to the surgical headgear, and a plurality of comfort layers stacked one upon the other on a second side of the padding layer. Each comfort layer includes a pressure sensitive adhesive layer proximate the padding layer, and a contact layer distal from the padding layer. The adhesive layer of each comfort layer faces the padding layer and the contact layer of each adhesive layer faces the away from the padding layer. A distal-most one of the comfort layers is removable from the padding assembly to expose a next one of the comfort layers.

In some embodiments, the padding layer may have a thickness between about ⅛ inch and about ½ inch. Each comfort layer may include a substantially waterproof barrier layer between the adhesive layer and the contact layer. The padding assembly may further include a padding envelope completely covering the padding layer and formed of an absorbent comfort material. The padding envelope may include disposal indicia providing instructions to dispose of the padding assembly, and the disposal indicia may be revealed upon removal of the comfort layer that is most proximate to the padding layer. Each contact layer may include removal indicia providing instructions to remove the contact layer prior to use.

In some embodiments, the attachment layer may completely cover the first side of the padding layer. The attachment layer may include a ridged backing layer covering the entire first side of the padding layer and at least partially imparting a curvature to the padding assembly. In some configurations the contact layer may be or include an absorbent comfort material, and/or the attachment layer may include at least one component of a two-component fastening system.

In some embodiments, each comfort layer may include a pull tab extending outwardly beyond at least one of the adhesive layer and the contact layer of the corresponding comfort layer. Each pull tab may be positioned on one end of the padding assembly, and adjacent pull tabs may be arranged in a staggered configuration such that one pull tab is offset relative to an immediately adjacent pull tab.

In other aspects, a surgical headgear padding assembly kit may include a plurality of padding assemblies, and at least some of the padding assemblies may have a length that is different than others of the padding assemblies.

In still other aspects, a padding assembly releasably attachable to surgical headgear may be provided, including a padding layer formed of a compliant solid state gel material and having a thickness between about ⅛ inch and ½ inch, a padding envelope completely covering the padding layer and formed of an absorbent comfort material, and a substantially rigid attachment layer coupled to the padding envelope on a first side of the padding layer. The attachment layer may include at least one component of a two-component fastening system and may be configured for attachment to the surgical headgear. The padding assembly may also include a plurality of comfort layers stacked one upon the other on a second side of the padding layer. Each comfort layer may include a pressure sensitive adhesive layer proximate the padding layer, a contact layer distal from the padding layer, a barrier layer between the adhesive layer and the contact layer, and a pull tab extending outwardly beyond at least one of the adhesive layer and the contact layer. The adhesive layer of each comfort layer may face the padding layer and the contact layer of each adhesive layer may face the away from the padding layer. A distal-most one of the comfort layers may be removable from the padding assembly to expose a next one of the comfort layers.

In some embodiments, the padding envelope may include disposal indicia providing instructions to dispose of the padding assembly, and the disposal indicia may be revealed upon removal of the comfort layer that is most proximate to the padding layer. In the same or other embodiments, each contact layer may include removal indicia providing instructions to remove the contact layer prior to use.

In some embodiments, each pull tab may be positioned on one end of the padding assembly, and adjacent pull tabs may be arranged in a staggered configuration such that one pull tab is offset relative to an immediately adjacent pull tab.

In still other aspects, A kit includes a surgical headgear including a headband, a crown support, and a plurality of attachment points provided on the headband and crown support. The kit also includes at least one padding assembly releasably attachable to the plurality of attachment points. The padding assembly includes a padding layer formed of a compliant solid state gel material, an attachment layer coupled to a first side of the padding layer and attachment members configured for attachment to the attachment points, and a plurality of comfort layers stacked one upon the other on a second side of the padding layer. Each comfort layer includes a pressure sensitive adhesive layer proximate the padding layer, and a contact layer distal from the padding layer. The adhesive layer of each comfort layer faces the padding layer and the contact layer of each adhesive layer faces the away from the padding layer. While the padding assembly is attached to the surgical headgear, a distal-most one of the comfort layers is removable from the padding assembly to expose a next one of the comfort layers.

### BRIEF DESCRIPTION OF THE DRAWINGS

FIG. 1 is a perspective view of an exemplary padding assembly.

FIG. 2 is an exploded perspective view of the padding assembly of FIG. 1.

FIG. 3 is a side view of the padding assembly of FIG. 1.

FIG. 4 is a section view taken along line 4-4 of FIG. 3

FIG. 5 is a perspective view of a surgical headgear suitable for use with the padding assembly of FIG. 1.

FIG. 6 is an enlarged view of an interior of the surgical headgear of FIG. 5.

Before any embodiments of the invention are explained in detail, it is to be understood that the invention is not limited in its application to the details of construction and the arrangement of components set forth in the following description or illustrated in the accompanying drawings. The invention is capable of other embodiments and of being practiced or of being carried out in various ways. Also, it is to be understood that the phraseology and terminology used herein is for the purpose of description and should not be regarded as limiting.

### DETAILED DESCRIPTION

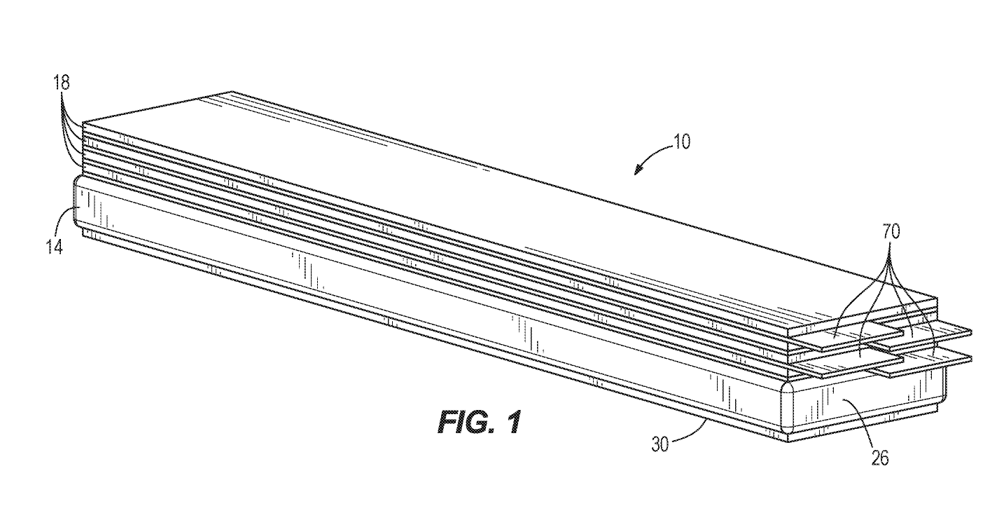

FIGS. 1-4 illustrate a padding assembly 10. Although various applications are possible, the illustrated padding assembly is particularly well suited for use as internal padding in surgical headgear such as face shields, head lamps, and the like. The padding assembly 10 includes a padding layer 14 formed of a compliant, resilient, padding material, and a plurality of removable comfort layers 18 attached to the padding layer 10 and stacked one upon the other. The comfort layers 18 are removable one after the other from the padding assembly 10 to, for example, expose a fresh comfort layer 18 after an outermost comfort layer 18 has become soiled. Although the illustrated padding assembly 10 is in the shape of an elongated rectangle, the construction techniques and assembly configurations described herein can be used to produce a padding assembly of substantially any shape to accommodate a given application.

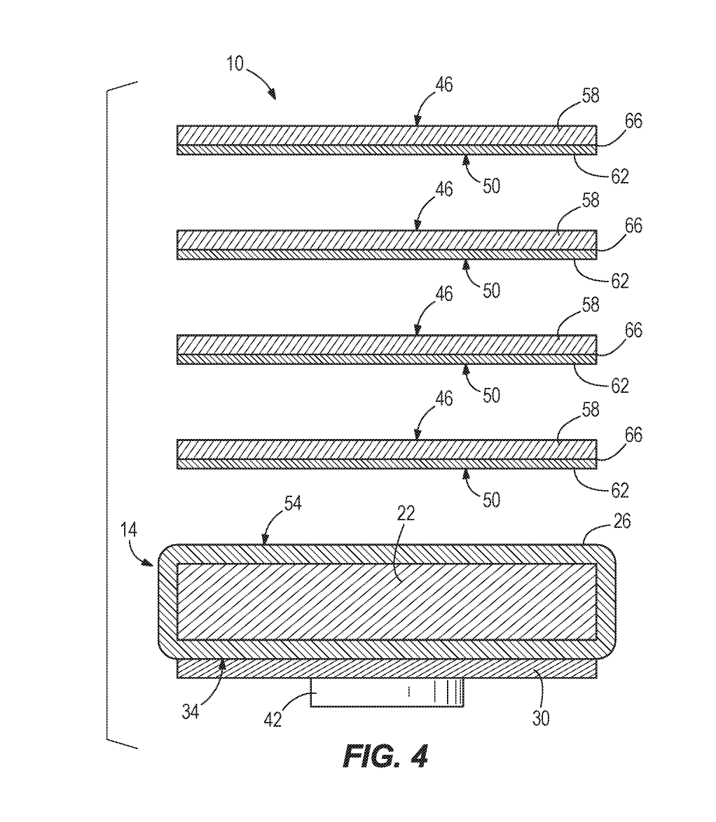

As best shown in FIG. 4, the padding layer 14 includes a core 22 having a generally rectangular cross section. In one exemplary construction, the core 22 is formed of a compliant, solid state gel material, such as SHOCKtec Gel available from Kemmler Products, Inc. of Mooresville, N.C. The illustrated padding layer 14 also includes a padding envelope or cover 26. The illustrated cover 26 completely surrounds the core 22 such that no portion of the core is exposed. In other configurations, the cover 26 may cover fewer than all surfaces of the core 22 or may be eliminated altogether. For embodiments in which the cover 26 covers less than all of the surfaces of the core 22, different materials or techniques may be used to prevent or reduce discomfort that might be caused by a user's skin or hair contacting untreated material of the core 22. By way of example only, when the SCHOCKtec Gel material mentioned above is cut to shape, the edges that are cut may be somewhat tacky. One known method for eliminating this tackiness when the surfaces are left exposed is to coat the surface with talcum powder or corn starch.

The cover 26 may be formed from a comfort material that is at least somewhat absorbent and provides a relatively soft, non-abrasive feel against the skin while still providing an adequate level of friction to prevent unwanted movement of the padding assembly 10 and the equipment to which the padding assembly is secured relative to the wearer. In various exemplary embodiments the comfort material may be or include any of the materials commonly used for surgical drape sheets, including 2 and 3 ply tissue, tissue/polyester blends, polyester/cellulose blends, and non-woven blends. In some embodiments the comfort material may provide both a relatively comfortable outer surface as well as a fluid resistant inner barrier layer.

The padding assembly 10 also includes an attachment layer 30 coupled to a first side 34 of the padding layer 14. In the illustrated configuration the attachment layer 30 is coupled to the cover 26. In embodiments where the cover 26 does not cover the first side 34 of the padding layer 14, the attachment layer 30 may be directly coupled to the core 22, for example by a suitable adhesive. In the illustrated embodiment, the attachment layer 30 includes a semi-rigid backing plate 38 that may be formed or molded into a predetermined shape (such as a curve) to impart the predetermined shape to the padding assembly 10 such that the shape of the padding assembly substantially corresponds to a shape of the article into which the padding assembly 10 will be installed. The illustrated attachment layer 30 also includes at least one attachment member 42, which may be in the form of one or more relatively small patch-like members or in the form of one or more larger members covering most or all of the backing plate 38. In some constructions the attachment member(s) 42 may be or include at least one component of a two-component fastening system, such as one half of a hook and loop style fastening system configured for securement to a corresponding hook and loop style fastening system provided on the article to which the padding assembly 10 is to be secured.

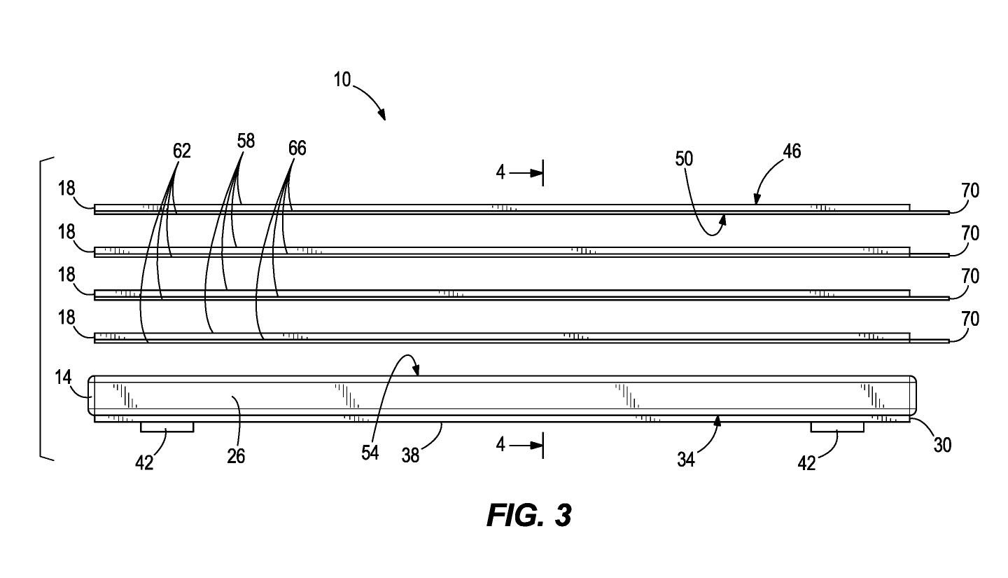

In other embodiments, the attachment layer 30 may be or include a single layer of material in which one side is secured to the core 22 (or to the cover 26, if the cover 26 covers the first side 34 of the padding layer 14) and the other side faces away from the first side 34 of the padding layer 14 and is exposed. In such embodiments the exposed side of the attachment layer 30 may be or include a hook and loop style fastening system configured for securement to a corresponding hook and loop style fastening system provided on the article to which the padding assembly 10 is to be secured. The attachment layer 30 formed of a single layer of material may completely cover the first side 34 of the padding layer 14. By way of example, and with reference to FIGS. 3 and 4, the embodiment just described would be similar to the embodiment shown in FIGS. 3 and 4 but with the attachment members 42 removed. In still other embodiments, the attachment layer 30 may include individual attachment elements spaced intermittently along the first side 34 of the padding layer 14.

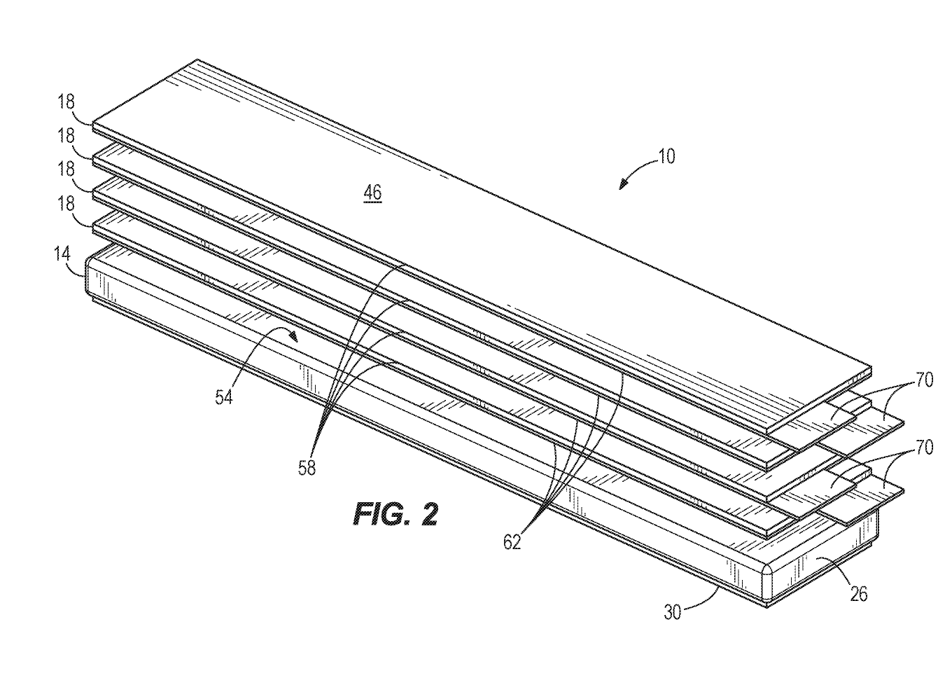

Each comfort layer 18 includes a comfort side 46 configured for direct engagement with a user, and an adhesive side 50 opposite the comfort side 46. The adhesive side 50 of each comfort layer 18 is configured to secure the comfort layer 18 to the comfort side 46 of an adjacent comfort layer 18 or, for the comfort layer 18 that is most proximate to the padding layer 14, to a second side 54 of the padding layer 14 that is opposite the first side 34 of the padding layer 14. Although the comfort layers 18 are shown spaced from one another and from the padding layer in FIGS. 2-4, it should be understood that the comfort layers 18 directly contact one another and the most proximate of the comfort layers 18 directly contacts the padding layer 14 as shown in FIG. 1.

Each comfort layer 18 includes at least a contact layer 58 defining the comfort side 46 and an adhesive layer 62 defining the adhesive side 50. In some embodiments, each comfort layer 18 may also include a barrier layer 66 positioned between the contact layer 58 and the adhesive layer 62 to define a substantially liquid impermeable barrier between the comfort side 46 and the adhesive side 50. Depending on the specific construction of the comfort layer 18, one or more additional layers may be provided between contact layer 58, the adhesive layer 62, and the barrier layer 66, for example to secure these layers to one another and/or to provide other desirable characteristics to the comfort layer 18. In the illustrated configuration the contact layer 58, adhesive layer 62, and barrier layer 66 are substantially the same shape and coextensive with one another. In alternative configurations one or more of the layers may not cover or be coextensive with one or more of the other layers. For example, it may be desirable for the barrier layer 66 to be secured to the contact layer 58 only near the ends of the comfort layer 18. In other alternative embodiments it may be desirable for the adhesive layers 62 to be discontinuous and present only near the ends of the comfort layer 18 such that adjacent comfort layers 18 are secured to one another only at their ends, thereby leaving the middle portions of the comfort layers 18 free to move with respect to one another. In other alternative embodiments the adhesive layers 62 may cover only the edges of the comfort layer 18 in a “picture frame” configuration to reduce the likelihood of the adhesive removing bits of fiber from the underlying comfort layer 18 when removed.

Each comfort layer 18 may also include a depending portion that defines a pull tab 70, which may be used to remove the respective comfort layer 18 from the padding assembly 10. The pull tab 70 of a given comfort layer 18 may be formed integrally with one or more of the layers 58, 62, 66, or may be a separate component that is secured to or between one or more of the layers 58, 62, 66. Each pull tab 70 is configured to extend outwardly beyond a perimeter of the layers 58, 62, 66 to facilitate grasping by a user to remove the associated comfort layer 18 from the padding assembly 10 without removing the other comfort layers 18. In the illustrated configuration, the pull tabs 70 are all positioned on one end of the padding assembly 10 but are arranged in a staggered or alternating pattern such that one pull tab 70 is not directly above the pull tab 70 for an adjacent comfort layer 18, thereby facilitating grasping by a user. In other configurations the pull tabs 70 may be spaced and/or positioned along other edges of the comfort layer 18 or on opposing edges of the comfort layer 18 as desired for a given application.

The contact layer 58 defines the contact surface 46 and is configured and selected to provide a comfortable surface against a user's skin. The adhesive layer 62 defines the adhesive side 50 and is configured and selected to provide a low-tack, pressure sensitive adhesive surface for securing each comfort layer 18 to the contact surface 46 of an adjacent comfort layer 18 or, in the case of the comfort layer 18 that is directly adjacent to the padding layer 14, to the second side 54 of the padding layer 14. The optional barrier layer 66 positioned between the contact layer 58 and the adhesive layer 62 may be configured to prevent sweat, dirt, and other contaminants from being transferred from an exposed comfort layer 18 to a comfort layer 18 positioned directly below the exposed comfort layer 18. When the padding assembly 10 is assembled and ready for use, the adhesive layer of each comfort layer 18 is proximate to the padding layer 14, and the contact layer 54 of each comfort layer 18 is distal from the padding layer 14.

In some embodiments, the contact layer 58 may be or include the comfort material described above with respect to the cover 26. In other embodiments a different material having the same or similar properties may also or alternatively be used to form the contact layer 58. In some embodiments, the barrier layer 66 may be integrally formed with the contact layer 58 as part of the comfort material. In other embodiments, the barrier layer 66 may be integrally formed with the adhesive layer 62. In one exemplary embodiment, the contact layer 58 may be or include one of the comfort materials described above, the adhesive layer 62 and the barrier layer 66 may be or include a transparent and waterproof low tack removable masking film, such as Frisket Film, available from Grafix Arts of Maple Heights, Ohio, and a spray adhesive, such as Krylon All Purpose Spray Adhesive #7010, available from Krylon Products Group of Cleveland, Ohio, may be used to secure the low tack removable masking film that makes up the adhesive layer 62 and the barrier layer 66 to the contact layer 58.

The comfort side 46 of each comfort layer 18 may include indicia instructing a user to remove the comfort layer 18 before use, thereby exposing a clean comfort layer 18. To prevent a user from unnecessarily removing the outermost comfort layer 18 during the first use of the padding assembly 10, an opaque release liner (not shown) may be provided covering the outermost comfort layer 18 and may also include indicia instructing the user to remove the release liner before the initial use. The release liner may also prevent contamination or soiling of the outermost comfort layer 18 during packaging and transit. The second side 54 of the padding layer 14 may also include indicia instructing the user to discard the padding assembly after use. This indicia would be revealed upon removal of the comfort layer 18 that is attached directly to the padding layer 14.

In alternative embodiments, the comfort layers 18 may be secured to one another in different ways. For example, the comfort layers 18 may be joined to one another by perforations running along at least one common edge. In these embodiments, rather than peeling away a layer that is secured by adhesive, a user would be tearing the outermost comfort layer 18 away from the comfort layer 18 immediately beneath along the perforations. In these embodiments the comfort layers 18 may be folded in an accordion fashion and/or joined to one another via perforations along two parallel or perpendicular edges. Pull tabs similar to those described above may still be provided to facilitate removal of one comfort layer 18 at a time.

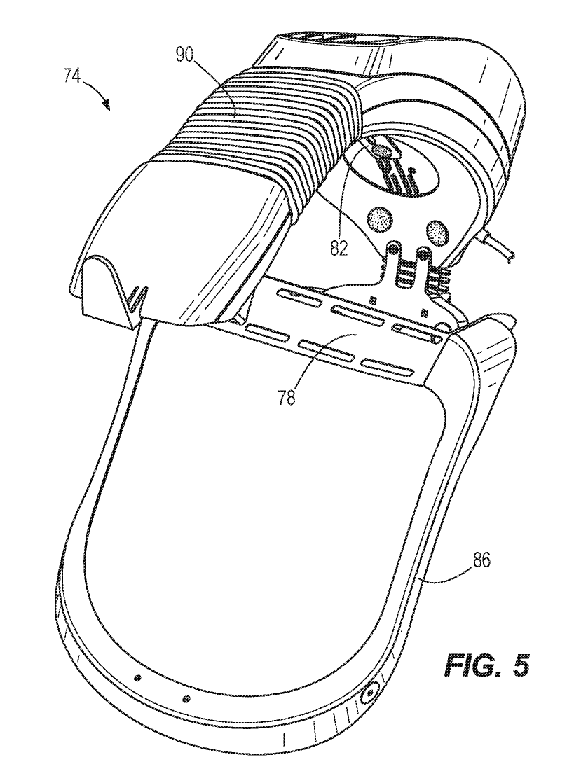

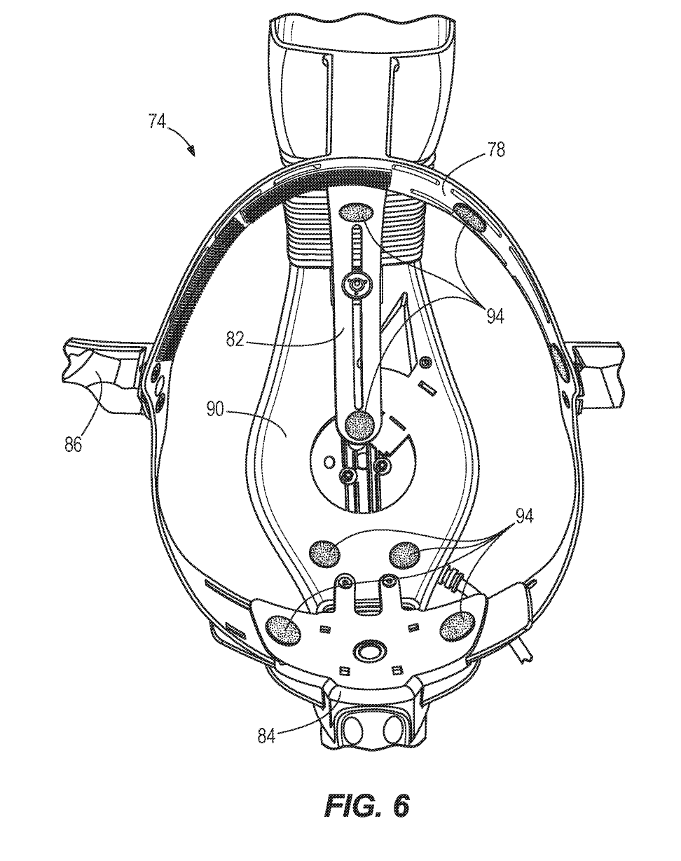

Referring also to FIGS. 5 and 6, an exemplary surgical headgear 74 with which one or more padding assemblies 10 may be used is illustrated. The headgear 74 includes a circumferential headband 78 and a crown support 82 extending in an arcuate path diametrically between front and rear ends of the headband 78. An adjustment mechanism 84 is positioned at the rear of the headband 78 and is operable to adjust the size of the headband 78 in a circumferential direction. A face shield support 86 extends downwardly from the headband 78 and a light and blower assembly 90 is mounted upon the crown support member 82 and is configured to illuminate and ventilate the area in front of the face shield support 86. It should be appreciated that the headgear 74 is merely one example of a type of headgear with which the padding assembly 10 may be used and that the padding assembly may also be used in combination with other types of surgical or non-surgical headgear.

As best shown in FIG. 6, the headgear 74 includes a plurality of attachment points 94 positioned on the headband 78 and the crown support 82. Although other configurations are possible, in the illustrated configuration each attachment point 94 comprises one half of a hook and loop attachment system. In this way, the attachment layer 30, which as discussed above may be or include one half of a hook and loop attachment system, may be used to secure the padding assembly 10 to the inside of the headgear 74. In embodiments where the attachment layer 30 includes attachment members 42, the attachment members 42 are aligned with the attachment points 94 on the headgear 74 and the padding assembly 10 is thereby secured to the headgear 74. In embodiments where the attachment layer 30 includes one half of a hook and loop attachment system extending along some or all of the first side 34 of the padding layer 14, the padding assembly 10 may be positioned in a variety of positions by securing the attachment layer 30 to the attachment points 94. In this regard a padding assembly 10 with an attachment layer 30 with hook and loop attachment extending along all or a relatively large portion of the padding layer 14 may permit greater variation in the specific location of the padding assembly 10 within the headgear 74 to accommodate a variety of wearers.

In exemplary configurations, padding assemblies 10 of varying lengths may be used in combination with one another to line the interior of the headgear 74 and to thereby provide comfortable support of the headgear 74 upon a user's head. By way of example, three relatively short padding assemblies 10 may be used to line the headband 78, one relatively short padding assembly 10 may be positioned transversely near the rear of the crown support 82, and one relatively long padding assembly 10 may be positioned along and aligned with the frontal portion of the crown support 82. It should be appreciated that different types or styles of headgear 74 may call for the use of padding assemblies 10 having different shapes and sized. In each case, a kit may be provided whereby padding assembles 10 of the appropriate shapes and sizes for a given type of headgear 74 are supplied as a single unit in a bag or other suitable packaging.

An exemplary sequence of use of a kit of padding assemblies 10 will now be described. A user opens the kit and secures the various padding assemblies 10 contained therein in the appropriate locations on the corresponding headgear by contacting the attachment layer 30 of each padding assembly 10 to the corresponding attachment members 42. After securing the padding assemblies 10, release liners, if present, are removed from each padding assembly to expose the outermost comfort layer 18. The headgear 74 may then be donned and used. During use the core 22 contributes to the user's overall comfort by providing resilience and padding against the wearer's head, while the comfort side 46 of the exposed comfort layer 18 contributes to the user's overall comfort by positioning a relatively soft and preferably absorbent material in close proximity to the wearer's head. When the headgear 74 is no longer needed it may be removed and set aside without further manipulation of the padding assemblies 10 by the user. Before the next use of the headgear 74, the user would use the pull tab 70 to remove the previously used outermost comfort layer 18 to expose the comfort layer 18 immediately beneath. With the barrier layer 66 positioned between the contact layer 58 of the first comfort layer 18 and the contact layer 58 of the second comfort layer 18, the comfort side 46 of the second comfort layer 18 will be substantially clean and sanitary upon removal of the first comfort layer 18. The headgear 74 may then be used as previously described.

Before each subsequent use of the headgear, the exposed comfort layer 18 is removed to expose the substantially clean and sanitary comfort layer 18 immediately beneath. For embodiments with a layer of comfort material covering the second side 54 of the padding layer 14, when the last comfort layer 18 is removed the padding assembly 10 may be used one more time and then discarded. In these instances the comfort material on the second side 54 of the padding layer 14 functions as the contact layer 58 and when the headgear 74 is no longer needed the padding assemblies 10 are removed from the headgear 74 and discarded or recycled. For embodiments that do not include a layer of comfort material over the second side 54 of the padding layer 14, when the headgear 74 is removed after using it with the last comfort layer 18 exposed the padding assemblies 10 are removed from the headgear 74 and discarded or recycled.

Various features of the invention are set forth in the following claims.

What is claimed is:

1. A padding assembly releasably attachable to surgical headgear, the padding assembly comprising:
a padding layer formed of a compliant solid state gel material;
an attachment layer coupled to a first side of the padding layer and configured for attachment to the surgical headgear; and
a plurality of comfort layers stacked one upon the other on a second side of the padding layer, each comfort layer including a pressure sensitive adhesive layer proximate the padding layer, and a contact layer distal from the padding layer,
wherein the adhesive layer of each comfort layer faces the padding layer and the contact layer of each adhesive layer faces the away from the padding layer, and wherein a distal-most one of the comfort layers is removable from the padding assembly to expose a next one of the comfort layers.

2. The padding assembly of claim 1, wherein the padding layer has a thickness between about ⅛ inch and about ½ inch.

3. The padding assembly of claim 1, wherein each comfort layer includes a substantially waterproof barrier layer between the adhesive layer and the contact layer.

4. The padding assembly of claim 1, further comprising a padding envelope completely covering the padding layer and formed of an absorbent comfort material.

5. The padding assembly of claim 4, wherein the padding envelope includes disposal indicia providing instructions to dispose of the padding assembly, and wherein the disposal indicia is revealed upon removal of the comfort layer that is most proximate to the padding layer.

6. The padding assembly of claim 1, wherein each contact layer includes removal indicia providing instructions to remove the contact layer prior to use.

7. The padding assembly of claim 1, wherein the attachment layer completely covers the first side of the padding layer.

8. The padding assembly of claim 1, wherein the attachment layer includes a ridged backing layer covering the entire first side of the padding layer and at least partially imparting a curvature to the padding assembly.

9. The padding assembly of claim 1, wherein the contact layer comprises an absorbent comfort material.

10. The padding assembly of claim 1, wherein the attachment layer includes at least one component of a two-component fastening system.

11. The padding assembly of claim 1, wherein each comfort layer includes a pull tab extending outwardly beyond at least one of the adhesive layer and the contact layer of the corresponding comfort layer.

12. The padding assembly of claim 11, wherein each pull tab is positioned on one end of the padding assembly, and wherein adjacent pull tabs are arranged in a staggered configuration such that one pull tab is offset relative to an immediately adjacent pull tab.

13. A surgical headgear padding assembly kit comprising a plurality of padding assemblies according to claim 1, at least some of the padding assemblies having a length that is different than others of the padding assemblies.

14. A padding assembly releasably attachable to surgical headgear, the padding assembly comprising:
a padding layer formed of a compliant solid state gel material and having a thickness between about ⅛ inch and ½ inch;
a padding envelope completely covering the padding layer and formed of an absorbent comfort material;
a substantially rigid attachment layer coupled to the padding envelope on a first side of the padding layer, the attachment layer include at least one component of a two-component fastening system and configured for attachment to the surgical headgear; and
a plurality of comfort layers stacked one upon the other on a second side of the padding layer, each comfort layer including a pressure sensitive adhesive layer proximate the padding layer, a contact layer distal from the padding layer, a barrier layer between the adhesive layer and the contact layer, and a pull tab extending outwardly beyond at least one of the adhesive layer and the contact layer,
wherein the adhesive layer of each comfort layer faces the padding layer and the contact layer of each adhesive layer faces the away from the padding layer, and wherein a distal-most one of the comfort layers is removable from the padding assembly to expose a next one of the comfort layers.

15. The padding assembly of claim 14, wherein the padding envelope includes disposal indicia providing instructions to dispose of the padding assembly, and wherein the disposal indicia is revealed upon removal of the comfort layer that is most proximate to the padding layer.

16. The padding assembly of claim 14, wherein each contact layer includes removal indicia providing instructions to remove the contact layer prior to use.

17. The padding assembly of claim 14, wherein each pull tab is positioned on one end of the padding assembly, and wherein adjacent pull tabs are arranged in a staggered configuration such that one pull tab is offset relative to an immediately adjacent pull tab.

18. A kit comprising:
a surgical headgear including a headband, a crown support, and a plurality of attachment points provided on the headband and crown support; and,
at least one padding assembly releasably attachable to the plurality of attachment points, the padding assembly including:
a padding layer formed of a compliant solid state gel material,
an attachment layer coupled to a first side of the padding layer and attachment members configured for attachment to the attachment points, and
a plurality of comfort layers stacked one upon the other on a second side of the padding layer, each comfort layer including a pressure sensitive adhesive layer proximate the padding layer, and a contact layer distal from the padding layer,
wherein the adhesive layer of each comfort layer faces the padding layer and the contact layer of each adhesive layer faces the away from the padding layer, and wherein while the padding assembly is attached to the surgical headgear, a distal-most one of the comfort layers is removable from the padding assembly to expose a next one of the comfort layers.
